(19) United States
(12) Patent Application Publication
(10) Pub. No.: US 2024/0165101 A1
(43) Pub. Date: May 23, 2024

(54) USE OF MEVIDALEN AND OTHER D1 POSITIVE ALLOSTERIC MODULATORS FOR SLOWING OF PARKINSON'S DISEASE PROGRESSION

(71) Applicant: Eli Lilly and Company, Indianapolis, IN (US)

(72) Inventors: Kevin Michael Biglan, Indianapolis, IN (US); Kjell Anders Ivan Svensson, Indianapolis, IN (US)

(21) Appl. No.: 18/549,693

(22) PCT Filed: Mar. 8, 2022
(86) PCT No.: PCT/US2022/019339

§ 371 Date: Sep. 8, 2023

Related U.S. Application Data
(60) Provisional application No. 63/158,460, filed on Mar. 9, 2021.

(51) Int. Cl. A61K 31/472

(52) U.S. Cl. CPC A61K 31/472

(57) ABSTRACT

The present invention relates to methods of treating and dosing regimens using Mevidalen, also described as 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone, and/or pharmaceutical compositions thereof, for slowing Parkinson's disease progression.

The present application claims the benefit of priority to U.S. Provisional Patent Application No. 63/158,460, filed on Mar. 9, 2021, the entire disclosure of which is incorporated by reference herein.

The present disclosure provides methods of using mevidalen, also described as 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone, and/or co-crystals and pharmaceutical compositions thereof, and/or other dopamine D1 positive allosteric modulators, for slowing the progression of Parkinson's Disease.

Parkinson's Disease is characterized by loss of dopamine-generating cells in a region of the brain known as the substantia nigra. However, the mechanisms responsible for the dopaminergic cell loss in Parkinson's Disease are unknown. PD undergoes six neuropathological stages, according to Braak staging, initially involving the deposition of intraneural inclusion bodies in the anterior olfactory nucleus and dorsal motor nucleus of the vagal nerve (stage 1), before advancing to denudation of melanoneurons in the substantia nigra (stage 3). In this middle stage patients start to have noticeable motor symptoms. Patients seeking consultation at the earliest clinical manifestation are, in fact, pathologically in the intermediate stages of their disorder, with perhaps 50% or more of the dopaminergic neurons degenerated in the substantia nigra.

Treatment of Parkinson's Disease focuses on increasing dopamine levels in the brain, and the various dopaminergic drugs used in treatment aim to either replace dopamine or prevent its degradation (See Treatment for the progression of Parkinson's disease. Calne, Donald, et al., The Lancet. Neurology (2005), 4(4), 206). A number of Parkinson's Disease symptoms arise from decreased dopamine levels and can be classified into motor and non-motor symptoms. The group of motor symptoms include i) resting tremor that affects, for example the arms, legs and jaw; ii) a slowness of movement or bradykinesia, and iii) lack of movement/rigidity or akinesia. In addition, the motor symptoms experienced by Parkinson's Disease sufferers also include problems with posture (instability thereof) and gait. Non-motor symptoms of Parkinson's Disease patients include neuropsychiatric symptoms, sleep disorders and wakefulness, and autonomic symptoms. Parkinson's Disease is a heterogeneous disorder with a clinical presentation that varies substantially from patient to patient. Current clinical guidelines (EMA 2012 guideline (EMA/CHMP/330418/2012 rev. 2) provide that the clinical diagnosis of Parkinson's Disease requires bradykinesia and at least one of the following: resting tremor, muscular rigidity and postural reflex impairment (core symptoms).

Parkinson's Disease is characteristically a progressive neurodegenerative disorder, and patients may have a long prodromal stage followed by the well-known clinical stages of disease. An understanding of etiopathogenesis of Parkinson's Disease has generated neuroprotective strategies aimed at early intervention, perhaps even in the prodromal phase, as an approach to alter the progression of the disease (Jankovic J, Tan E K. J Neurol Neurosurg Psychiatry 2020; 91:795-808). The progression of Parkinson's Disease is apparent from the response of the patient to dopaminergic therapy over time. In the initial stages of the disease the principal symptoms such as tremor, bradykinesia, and rigidity can usually be ameliorated by dopaminergic therapies such as dopamine agonists, monoamine oxidase inhibitors or levodopa. None of these conventional treatments are considered to slow the progression of Parkinson's Disease but can help patients control at least some motor symptoms. Parkinson's Disease patients are said to be in the ON state when they are free, or largely free, of the symptoms described above. In contrast, Parkinson's Disease patients are said to be in the OFF state when they are not in the ON state, for example when they exhibit Parkinson's Disease symptoms.

In the initial stage of disease, the symptoms of Parkinson's Disease may be ameliorated by treatment with dopamine agonists, monoamine oxidase inhibitors or levodopa. None of these conventional treatments are considered to slow disease progression, but all can help at least some patients control the symptoms of the disease, such as tremor. After this initial disease stage, the efficacy of the dopaminergic therapy reduces and “wearing off” or “end of dose” deterioration (such as motor fluctuations) occurs in most patients, as well as dyskinesia (the phenomenon of drug-induced involuntary movements including chorea and dystonia). Motor fluctuations refer to a situation where the patients fluctuate between being in the ON state and the OFF state, with a tendency, as the Parkinson's Disease progresses, to have an increase in time of being in the OFF state (‘OFF time’) and a decrease in time of being in the ON state (‘ON time’). These can be dose-dependent and more predictable, or non-dose-dependent. Because the effectiveness of levodopa can appear to decrease over longer periods of time, certain physicians prefer to commence treatment of Parkinson's Disease with a dopamine agonist and/or monoamine oxidase inhibitor, reserving treatment with levodopa until later in the treatment sequence. Levodopa is normally used together with an AADC inhibitor such as carbidopa or benserazide in order to reduce its peripheral metabolism and so reduce the dose required. It may also be used together with a COMT inhibitor, such as entacapone, to further reduce its peripheral metabolism and so further reduce the dose of levodopa required. These approaches are limited, and there remains a need for alternative and improved therapies to slow the progression of Parkinson's disease. Recently discovered dopamine D1 positive allosteric modulators may represent a novel approach to slow Parkinson's disease progression.

The dopamine receptor D1 subtype (D1) is the most abundant dopamine receptor in the central nervous system and plays an important role in multiple CNS functions. For many years the modulation of dopamine signaling in dopaminergic CNS disorders has been attempted with direct D1 receptor agonists, but various D1 agonist agents have achieved very limited success as lack of efficacy, safety, tolerability, including notably unacceptable adverse effects, have limited the utility of such agents. In addition, D1 agonists have bell-shaped dose response curves on cognitive endpoints which complicates and confounds clinical use. Thus, prior attempts to develop clinically useful direct D1 receptor agonists have been largely unsuccessful due to receptor desensitization, poor ADME/PK properties, and dose limiting side effects such as hypotension. Direct acting dopamine therapies are also limited in effectiveness due in part to high dose associated cognition impairment, seizure risk, and tolerance development.

Mevidalen is a dopamine D1 receptor Positive Allosteric Modulator (D1 PAM) and represents a potential first-in-class agent for slowing the progression of Parkinson's disease. Mevidalen (CAS Registry No. 1638667-79-4) can be described chemically as 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone, and can be structurally represented as:

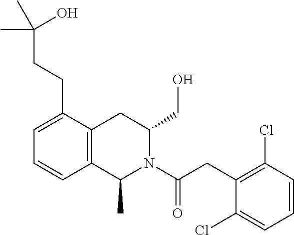 [SMILES: C[C@H]1c2cccc(CCC(C)(C)O)c2C[C@H](CO)N1C(=O)Cc1c(Cl)cccc1Cl]

Useful forms of mevidalen include a crystalline form (See WO 2017/070068), and a co-crystalline form comprising 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone and 4-hydroxybenzoic acid (CAS Registry No. 1638669-32-5) (See WO 2014/193781). As a positive allosteric modulator, also called a “potentiator” of the dopamine D1 receptor subtype, mevidalen is highly selective for D1. Mevidalen shows very weak direct agonism of the D1 receptor, and is active only in the presence of dopamine, and believed to be dependent on endogenous tone and subject to normal feedback control. Thus, mevidalen represents an innovative pharmacological agent and approach to modulating D1 signaling pathways for slowing the progression of Parkinson's disease where D1 signaling may be deficient, resulting in neuronal cell dysfunction and/or cell loss.
Mevidalen has a mechanism of action that differs from other dopaminergic agents such as the direct D1 receptor agonists. Mevidalen binds to a newly discovered allosteric binding site on intracellular loop 2 of the D1 receptor, where it increases the affinity of dopamine for the D1 receptor. Due to the complexity of dopaminergic signaling in normal physiology and clinical disease, and the lack of clinical pharmacological guidance from D1 orthosteric agonists, there remains an important unmet need for new methods of slowing the progression of Parkinson's disease using mevidalen and other dopamine D1 positive allosteric modulators. In particular there remains an unmet need for methods of slowing the progression of Parkinson's disease with dopamine D1 positive allosteric modulators, such as mevidalen, which provide a combined profile of effective, safe, and clinically tolerable pharmacological effects. Summary of Invention:

The D1 PAM mevidalen has now been evaluated in a Phase 2 clinical study (referred to as PRESENCE (NCT03305809)) in patients to assess the safety and efficacy in patients with mild-to-moderate Lewy Body Dementia (Parkinson's disease dementia—PDD or Dementia with Lewy Bodies—DLB) compared with placebo. For prespecified outcomes across measures of motor and non-motor symptoms, mevidalen demonstrated statistically significant and clinically meaningful benefits over the 12-week study. For example, mevidalen treatment resulted in significant improvement in global functioning (ADCS-CGIC). These observations, along with certain neuronal responses to D1 PAM exposure, led to the conception that treatment of early-stage Parkinson's patients may slow disease progression. In particular, dopamine D1 receptors are involved in synaptic plasticity impacting the ability of neuronal synapses to strengthen or weaken over time. Enhanced activation of dopamine D1 receptors by D1 PAM's of the present disclosure is believed to facilitate plasticity (neurogenesis) via enhanced neurite outgrowth, and increased numbers of dendritic spines and synapses. This response is believed to have the potential to delay neuronal dysfunction, and/or neuronal cell death in early-stage Parkinson's patients. Taken together, these data support the potential for chronic D1 PAM treatment to have long term disease modifying effects that could positively affect the course of the Parkinson's disease progression.

The present disclosure provides methods of using mevidalen, and/or pharmaceutical compositions thereof, and/or other dopamine D1 positive allosteric modulators, for use in slowing the progression of Parkinson's disease. As used herein, slowing the progression of Parkinson's disease refers to effects where the progression is delayed by a period of time.

The present disclosure provides means for slowing the progression of Parkinson's disease. As used herein, “slowing the progression of Parkinson's disease means inhibiting, delaying, stopping, any of the signs and symptoms of Parkinson's disease progression, to any significant degree, as evidenced by one or more the endpoints for signs and symptoms of Parkinson's disease which are known to the skilled artisan and/or described herein. Methods of measuring these endpoints, and changes therein, are likewise known to the skilled artisan and/or described herein, and improvements in these endpoints may be on the order for 5 percent, 10 percent, 15 percent, 20 percent, or more, at a given point in time of the disease course, and/or as evidence by a delay in progression of weeks, or months or years. For patients treated according to the methods of the present embodiments, the progression of Parkinson's disease may be delayed by a period of at least one month relative to patients who have not received the treatment of the invention, preferably by at least 3 months, or at least 4 months, or at least 5 months, or at least 6 months, or at least 7 months, or at least 9 months or at least one year.

In an embodiment the present disclosure provides a method of slowing or delaying progression of Parkinson's disease in a patient in need thereof, but previously untreated for Parkinson's disease, which comprises administering an effective dose of mevidalen to said patient.

In an embodiment the present disclosure provides the use of mevidalen, or a D1 PAM described herein, for slowing or delaying progression of Parkinson's disease in a patient diagnosed with, but previously untreated for, Parkinson's disease, comprising administering an effective dose of mevidalen, or a D1 PAM described herein, to said patient.

In an embodiment the present disclosure provides the use of mevidalen, or a D1 PAM described herein, for slowing the progression of idiopathic Parkinson's disease, with or without combination treatment with levodopa, over the course of the disease, including through to late stages when the effect of levodopa wears off or becomes inconsistent and fluctuations of the therapeutic effect occur (end of dose or ON-OFF fluctuations).

As used herein, a patient previously untreated for Parkinson's disease includes those PD patients who have not previously taken medication for treatment of PD. Medication for treatment of PD includes dopamine agonists, dopamine precursors such as levodopa, COMT inhibitors, MAO-B inhibitors, and anti-cholinergics. Before commencing treatment according to the embodiments of the present disclosure such patients may experience a mean daily OFF time of up to 6 hours, for example up to 1 hour, up to 2 hours, up to 3 hours, up to 4 hours, or up to 5 hours per day.

Before commencing treatment according to the embodiments of the present disclosure such patients may have had a PD diagnosis for up to 9 years, for example up to 1 month, up to 2 months, up to 3 months, up to 4 months, up to 6 months, up to 12 months, up to 2 years, up to 3 years, up to 4 years, up to 5 years, up to 6 years, up to 7 years or up to 8 years.

Before commencing treatment according to the embodiments of the present disclosure the patients may experience motor symptoms, and/or motor complications, such as motor fluctuations with or without dyskinesias, for up to 4 years, for example up to 1 month, up to 2 months, up to 3 months, up to 4 months, up to 6 months, up to 12 months, up to 2 years, or up to 3 years.

According to Movement Disorder Society-PD (MDS-PD) criteria, the clinical diagnosis of PD has centered on a defined motor syndrome (Parkinsonism), based on three cardinal motor symptoms (MS), such as bradykinesia, rigidity, and resting tremor. However, non-motor symptoms (NMS), which include insomnia, depression, anxiety, cognitive decline, apathy, neuropsychiatric disturbances, and autonomic dysfunctions, can be present at the onset and during disease progression. Therefore, PD increasingly compromises quality of life and activities of daily living, although a high variability in clinical presentation and in disease progression can be observed among people affected.

Thus, the Hoehn and Yahr scale is commonly used to compare groups of patients and to provide an indicative assessment of disease progression, ranging from stage I (only unilateral involvement) to stage V (wheelchair bound or bedding). In an early stage, symptoms are usually mild and unilateral with a complete response to treatment. Although symptoms tend to progress and motor symptoms affect the contralateral side, at first drug response is commonly effective. During the course of the disease, treatment response decreases, and anti-parkinsonian drugs can potentially induce side effects. After a prolonged disease duration, patients can develop several NMS for which current treatments are limited. Previous studies have reported that the mean duration of disease ranges from 6.9 to 14.3 years and the onset of dementia is the highest predictor of increased mortality.

Both clinical features of PD, motor and non-motor, are described herein in association with disease progression. Early stage may include: Motor Symptoms, REM Sleep Behavior Disorder (RBD), Constipation, Anxiety, Depression, Impulse Control Disorder. Advanced stage may include: Motor Symptoms, Cognitive deficits, Apathy, Psychotic disturbances, Orthostatic hypotension, Urinary dysfunction. Complicated Stage may include: Motor fluctuations, Dyskinesia, Super-off. (See for example, C. Carrrini, et al., A Stage-Based Approach to Therapy in Parkinson's Disease. Biomolecules 2019, 9, 388).

In the Early Stage several non-motor symptoms and signs of PD can be observed in the very early stages of disease. According to staging system of Braak, pathological process of PD may not begin in substantia nigra pars compacta. Synuclein deposition seems to firstly involve the anterior olfactory nucleus and dorsal motor nucleus of the vagus. A further subsequent finding suggests that peripheral autonomic ganglia and unmyelinated lamina-1 spinal cord neurons may also be involved in the initial pathological phases. These findings are consistent with PD typical early non-motor features, such as olfactory dysfunction, rapid eye movement (REM) sleep behavior disorder (RBD), constipation, anxiety, and depression. The quantitative assessment of clinical symptoms and progression of the course is an essential component of any therapeutic trial in PD. Parkinson's disease is known to have disease variability, thus symptom severity categorization can be based on a holistic evaluation, including neurologic examination and a detailed assessment of how symptoms affect daily functions and quality of life. Preferably, for the embodiments disclosed herein, an early-stage Parkinson's patient is one diagnosed as Hoehn & Yahr II, and off medications.

During progression of PD, in the Advanced Stage, the beneficial effects of early-stage therapies may reduce because of dopaminergic progressive neuronal loss, and management of MS becomes more complicated. Disabling manifestations of this advanced state include worsening of balance, falls, increasing compromise of gait, and speech disturbances. Another common symptom in advanced PD is represented by dystonia. Dystonia usually follows 1-dopa therapy administration and it may present with several onset patterns: during wearing off, at peak dose, or with biphasic timing. Approximately 30% of PD patients, treated with 1-dopa, tend to present “off-dystonia”, especially in the morning before the first 1-dopa dose. Differently from other patterns, off-dystonia is commonly painful, and the foot seems to be the main site of pain. By contrast, peak-dose dystonia tends to involve the neck, face, and upper limbs, whereas dystonia occurring as part of diphasic dyskinesia seems to mainly involve lower limbs. Dystonia, which typically presents after years of disease, rarely occurs in an early stage, usually related to young-onset PD and to autosomal recessive genetic parkinsonism forms, such as PARK-PARKIN (PARK2) and PARK-SNCA (PARK1) mutations. Nonetheless, several non-motor features may appear in the advanced stage, such as hallucinations, psychosis, dysautonomia, mood disorders, and dementia. Some NMS typically debut years before MS. However, late stages of PD are featured by adjunctive NMS, which are slighter or less common in early phases.

Complicated Stage may include motor fluctuations which generally develop after 4-6 years of therapy and affect about 50% of PD patients. Wearing-off is the most common type, but other motor complications, such as dose failure, beginning of dose worsening, end-of-dose rebound, freezing of gait, and Levodopa-induced dyskinesia (LID), can also develop during disease progression. Several therapeutic strategies have been adopted to reduce frequency and duration of the so-called off-periods (defined by recurrence of symptoms or lack of 1-dopa effects) but none of these drugs are able to completely control motor fluctuations and their side effects may limit optimal dosage.

Methods of assessing PD progression and the signs and symptoms thereof are well known the skilled artisan and described for example in the DSM-5 and other well-known diagnostic references, such as those described herein. Further, when evaluating the efficacy of therapies for Parkinson's disease progression, treated patients can be referred to as being in the ON or OFF states. Therapeutic benefit is thus established by an increase in ON time and/or a reduction in OFF time. An increase in ON time provides an increased duration of relief from symptoms. A decrease in OFF time provides for reduced periods of time in which PD patients undergoing therapy exhibit Parkinson's disease symptoms. Measure of ON and OFF time are conventionally established by observation, with the patient and/or physician keeping a diary of symptoms and their timings. The observed ON and OFF times in a treatment group can be compared with those in alternative treatment or placebo treated groups as appropriate to the clinical context.

Methods used for evaluations of ON and OFF time and other symptoms include the Unified Parkinson's Disease Rating Scale or UPDRS (see Fahn S. Unified Parkinson's disease rating scale. In Fahn S, Marsden C D, Goldstein M, and Calne D B (eds) Recent Developments in Parkinson's Disease. McMillan, 1987, New York) to follow the longitudinal course of PD and the MDS (Movement Disorder Society)-UPDRS (See Goetz, et al (1 Jan. 2007). “Movement Disorder Society-sponsored revision of the Unified Parkinson's Disease Rating Scale (MDS-UPDRS): Process, format, and clinimetric testing plan”. Movement Disorders 22 (1): 41-47). As known to the skilled artisan, various other approaches can be used as deemed appropriate to establish clinical response and/or are described herein. Non-limiting examples of the approaches that might be applicable are PDQ-39 (Jenkinson et al, The Parkinson's disease questionnaire. User manual for the PDQ-39, PDQ-8 and PDQ Summary Index. Oxford: Health Services Research Unit, Department of Public Health, University of Oxford, 1998), NMSS (Chaudhuri et al., “The metric properties of a novel non-motor symptoms scale for Parkinson's disease: results from an international pilot study,” Movement Disorders, vol. 22, no. 13, pp. 1901-1911, 2007), PDSS (Chaudhuri et al. The Parkinson's disease sleep scale: A new instrument for assessing sleep and nocturnal disability in Parkinson's disease. J Neurol Neurosurg Psychiatry 2002; 73:629-35) Hoehn and Yahr staging and Schwab and England ADL scales http://neurosurgery.mgh.harvard.edu/functional/pdstages.htm). Safety assessments such as mMIDI (Grant JE. Impulse control disorders: A clinician's guide to understanding and treating behavioral addictions. New York: W.W. Norton & Company; 2008) and C-SSR (Posner et al. Columbia-Suicide Severity Rating Scale. Am J Psychiatry, 2011 168(12) 1266-1277 can also be used to establish clinical endpoints.

Parkinson' disease patients also experience, in the course of their treatment, dyskinesia, which refers to a state where drug-induced involuntary muscle movements occur. Dyskinesia is not thought to be a symptom of Parkinson' disease itself but instead a side effect of the drugs used for the treatment of symptoms. Dyskinesia, for example resulting from high levels of levodopa, can occur during the ON state when the normal Parkinson' disease symptoms are otherwise under control. Levodopa-induced dyskinesia appears in patients who have taken levodopa for a prolonged period of time and generally occurs during the ON state although in late-stage disease, dyskinesia can occur in OFF state.

Dyskinesia can be categorized into three main types. The most common form is peak-dose dyskinesia that occurs at peak levodopa levels and can be ameliorated by reducing the levodopa dose. A second form is diphasic dyskinesia occurring when levodopa levels are rising or falling and can also be ameliorated by reducing the levodopa dose. A third form of dyskinesia is OFF-time dystonia, these are sustained muscle contractions that cause, for example, twisting and repetitive movements or abnormal postures. These OFF-time dystonia correlate with akinesia (inability to initiate movement) when levodopa levels are low and are treatable with levodopa. A major challenge in the treatment of Parkinson' disease is to improve the patient's ON time (and/or conversely reduce the OFF time) without increasing the dyskinesia associated with therapy. Late-stage Parkinson' disease patients, classified as those patients who, after the initiation of levodopa therapy may i) suffer from an insufficient control of symptoms despite treatment with levodopa and/or ii) may suffer from motor complications such as motor fluctuations (which can be dose dependent or non-dose dependent) and dyskinesia. Delayed-ON is a prolongation of the time required for an anti-Parkinsonian drug effect to appear. Dose-dependent (or predictable) motor complications are related to the time of dosing e.g. peak dose dyskinesias, end of dose (or wearing off) deterioration and biphasic dyskinesias. For less predictable motor complications (e.g. paroxysmal ON-OFF phenomenon, freezing), therapy intends to reduce the duration and/or intensity of OFF state. Hence, the main efficacy variable can be the decrease in the number, duration and/or intensity of OFF state. It should also be clear to what extent ON-time with dyskinesia and ON-time without dyskinesia is increased. In highly advanced PD, patients may suffer from severe and highly unpredictable and rapid motor fluctuations.

Therapies to slow disease progression could either prevent or postpone late motor complications or fluctuations, and/or delay disease progression. Preferably a treatment would slow, stop, or substantially reduce, further neurodegeneration, and delay disease progression. No pharmacotherapy currently exists that has shown a relevant delay in disease progression. For early untreated Parkinson's disease (de novo) patients a clinical goal to be achieved is to slow the progression of motor symptoms as assessed by change in UPDRS. For stable treated Parkinson's disease patients, a clinical goal to be achieved is to slow further decline of motor impairment, prevent progression of disability, and prevent motor and non-motor complications. Key outcome measurements for this stage could be the emergence of so-called axial symptoms: e.g. freezing of gait, loss of balance or Hoehn and Yahr stage III (“bilateral disease: mild to moderate disability with impaired postural reflexes; physically independent” or “mild to moderate bilateral disease; some postural instability; physically independent”).

For patients with advanced PD a clinical goal to be achieved is the prevention of disability. Clinical endpoints in this patent group are wide-ranging and include reductions in autonomic failure or falls, reduction of cognitive symptoms and possibly ‘time to’ dementia and ‘time to’ nursing home placement. The present disclosure provides embodiments wherein mevidalen or other D1 PAM's described herein are administered to a patient in need thereof to slow or delay the patents onset of any of the above stages of Parkinson's disease for any significant period of time, including weeks, months or years, as described herein.

### Mevidalen and Other D1 PAM's:

In the methods of treatment of the present disclosure, and as used herein, mevidalen is 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone in any form, and includes crystalline and co-crystalline forms thereof, in particular the benzoic acid co-crystalline form, and/or pharmaceutical compositions comprising these agents. The present disclosure provides methods for use of mevidalen to slow the progression of Parkinson's disease, in a patient in need thereof, comprising administering to said patient a dose of about 5 mg to about 60 mg, up to a maximum total dose of 60 mg per day, of mevidalen, or pharmaceutical composition thereof. Preferably the patient is in the prodromal-stage or early-stage of Parkinson's disease. The present disclosure further provides a method for use of mevidalen for slowing the progression of Parkinson's disease, in a patient in need thereof, comprising administering to said patient a dose of about 10 mg to about 50 mg, up to a maximum total dose of 50 mg per day, of mevidalen, or a pharmaceutical composition thereof. Methods of treatment using mevidalen, and/or other D1 PAM agents described herein, provide a novel approach to slow the progression of Parkinson's disease, without the side effects associated with other Parkinson's treatments.

Mevidalen, has been studied in a Phase 2 clinical study for Parkinson's Disease Dementia (PRESENCE, NCT03305809) and was discovered to have statistically significant and meaningful benefits for motor and non-motor symptoms. The PRESENCE study, having a summary description provided in Example 1, has resulted in the discovery that mevidalen when used according to the methods and dosing regimens of the present disclosure may induce a surprising and marked slowing or delay in the progression of Parkinson's disease. Thus, when used according to the present methods of treatment and dosing regimens, mevidalen provides a means to improve dopamine D1 signaling in a manner that provides an effective, safe, and clinically tolerable therapeutic regimen slowing the progression of Parkinson's disease, preferably in early-stage patients.

The present disclosure provides certain clinical dosing regimens for the chronic daily administration of mevidalen such that the patient suffering from early stages of Parkinson's disease will have relief of the signs and symptoms of Parkinson's disease progression, while avoiding other D1 PAM effects that would preempt these clinical benefits. In addition, the present disclosure provides for the chronic daily administration of mevidalen such that patient suffering from prodromal or early stages of Parkinson's disease will further be able to employ either lower or higher doses of mevidalen within the regimens of the present disclosure, such that effective slowing of disease progression is achieved for the individual patient while undesirable effects are avoided. Generally, the dosing regimens of the present disclosure provide the means for patients to benefit from D1 PAM activity, while avoiding certain undesired adverse cardiovascular activities that have been observed clinically and may represent on-target pharmacology for D1 PAMs as a class. Accordingly, the present disclosure provides dosing regimens for oral daily administration of mevidalen to a patient having early-stage Parkinson's disease, using certain doses of mevidalen which are described in detail below.

In an embodiment, the present disclosure provides a method for use of mevidalen in the slowing of Parkinson's disease progression, in a patient in need thereof, comprising administering to said patient a dose of about 5 mg to about 60 mg, up to a maximum total dose of 60 mg per day, of mevidalen, or pharmaceutical composition thereof.

In an embodiment, the present disclosure provides a method for use of mevidalen in the slowing of Parkinson's disease progression, in a patient in need thereof, comprising administering to said patient a dose of about 5 mg to about 60 mg, up to a maximum total dose of 60 mg per day, of mevidalen, or pharmaceutical composition thereof.

In an embodiment, the present disclosure provides a dopamine D1 positive allosteric modulator, or a pharmaceutically acceptable salt or co-crystal thereof, for use in the slowing of Parkinson's disease progression.

In an embodiment, the present disclosure provides a dopamine D1 positive allosteric modulator, or a pharmaceutically acceptable salt or co-crystal thereof, for use in the slowing of Parkinson's disease progression.

In an embodiment, the present disclosure provides a dopamine D1 positive allosteric modulator, or a pharmaceutically acceptable salt or co-crystal thereof, for use in the slowing of Parkinson's disease progression, wherein the patient is previously untreated for Parkinson's disease.

In an embodiment, the present disclosure provides a dopamine D1 positive allosteric modulator, or a pharmaceutically acceptable salt or co-crystal thereof, for use in in the slowing of Parkinson's disease progression, wherein the patient is diagnosed with, but previously untreated for, Parkinson's disease.

In an embodiment, the present disclosure provides a dopamine D1 positive allosteric modulator, or a pharmaceutically acceptable salt or co-crystal thereof, for use in in the slowing of Parkinson's disease progression, wherein the patient is in the prodromal-stage of Parkinson's disease.

In an embodiment, the present disclosure provides a dopamine D1 positive allosteric modulator, or a pharmaceutically acceptable salt or co-crystal thereof, for use in in the slowing of Parkinson's disease progression, wherein the patient is in the early-stage of Parkinson's disease.

In an embodiment, the present disclosure provides a dopamine D1 positive allosteric modulator, or a pharmaceutically acceptable salt or co-crystal thereof, for use in in the slowing of Parkinson's disease progression, wherein the patient is in the advanced-stage of Parkinson's disease.

In an embodiment, the present disclosure provides mevidalen, or a pharmaceutically acceptable co-crystal thereof, for use according any of the embodiments above, wherein the mevidalen, or a pharmaceutically acceptable co-crystal thereof, is orally administered daily in a dose of 5 to 60 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 10 to 50 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose selected from the group consisting of 10 mg, 15 mg, 20 mg, 25 mg, 30 mg, 35 mg, 40 mg, 45 mg, and 50 mg, per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 50 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 45 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 40 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 35 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 30 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 25 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 20 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 15 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is orally administered daily in a dose of 10 mg per dose.

In an embodiment, the present disclosure provides mevidalen, or a pharmaceutically acceptable co-crystal thereof, for use according to any of the embodiments above, wherein the mevidalen, or a pharmaceutically acceptable co-crystal thereof, is administered simultaneously, separately, or sequentially in combination with levodopa and/or deep brain stimulation.

In an embodiment, the present disclosure provides a method of slowing of Parkinson's disease progression, in a patient in need thereof, comprising administering to said patient a dopamine D1 positive allosteric modulator, or salt or co-crystal thereof.

In an embodiment, the present disclosure provides a method of slowing of Parkinson's disease progression, in a patient in need thereof, comprising administering to said patient a dopamine D1 positive allosteric modulator, or salt or co-crystal thereof, wherein the patient is in the prodromal stage of Parkinson's disease.

In an embodiment, the present disclosure provides a method of slowing of Parkinson's disease progression, in a patient in need thereof, comprising administering to said patient a dopamine D1 positive allosteric modulator, or salt or co-crystal thereof, wherein the patient is in the Early stage of Parkinson's disease.

In an embodiment, the present disclosure provides a method of slowing of Parkinson's disease progression, in a patient in need thereof, comprising administering to said patient a dopamine D1 positive allosteric modulator, or salt or co-crystal thereof, wherein the patient is in the Advanced stage of Parkinson's disease.

In an embodiment, the present disclosure provides a method according to any of the method embodiments above, wherein the mevidalen, or a pharmaceutically acceptable co-crystal thereof, is orally administered daily in a dose of 5 to 60 mg per dose.

In an embodiment, the present disclosure provides a method according to any of the method embodiments above, wherein the mevidalen, or a pharmaceutically acceptable co-crystal thereof, is orally administered daily in a dose of 10 to 50 mg per dose.

In an embodiment, the present disclosure provides a method according to any of the method embodiments above, wherein the mevidalen, or a pharmaceutically acceptable co-crystal thereof, is orally administered daily in a dose selected from the group consisting of 10 mg, 15 mg, 20 mg, 25 mg, 30 mg, 35 mg, 40 mg, 45 mg, and 50 mg, per dose.

In an embodiment, the present disclosure provides a method according to any of the method embodiments above, wherein the mevidalen, or a pharmaceutically acceptable co-crystal thereof, is orally administered daily in a dose of 50 mg per dose.

In an embodiment, the present disclosure provides a method according to any of the method embodiments above, wherein the mevidalen, or a pharmaceutically acceptable co-crystal thereof, is orally administered daily in a dose of 45 mg per dose.

In an embodiment, the present disclosure provides a method according to any of the method embodiments above, wherein the mevidalen, or a pharmaceutically acceptable co-crystal thereof, is orally administered daily in a dose of 40 mg per dose.

In an embodiment, the present disclosure provides a method according to any of the method embodiments above, wherein the mevidalen, or a pharmaceutically acceptable co-crystal thereof, is orally administered daily in a dose of 35 mg per dose.

In an embodiment, the present disclosure provides a method according to any of the method embodiments above, wherein the mevidalen, or a pharmaceutically acceptable co-crystal thereof, is orally administered daily in a dose of 30 mg per dose.

In an embodiment, the present disclosure provides a method according to any of the method embodiments above, wherein the mevidalen, or a pharmaceutically acceptable co-crystal thereof, is orally administered daily in a dose of 25 mg per dose.

In an embodiment, the present disclosure provides a method according to any of the method embodiments above, wherein the mevidalen, or a pharmaceutically acceptable co-crystal thereof, is orally administered daily in a dose of 20 mg per dose.

In an embodiment, the present disclosure provides a method according to any of the method embodiments above, wherein the mevidalen, or a pharmaceutically acceptable co-crystal thereof, is orally administered daily in a dose of 15 mg per dose.

In an embodiment, the present disclosure provides a method according to any one of the methods above, wherein the mevidalen, or a pharmaceutically acceptable co-crystal thereof, is orally administered daily in a dose of 10 mg per dose.

In an embodiment, the present disclosure provides a method of slowing or delaying the progression of Parkinson's disease in a patient in need thereof, comprising administering to said patient a dopamine D1 positive allosteric modulator, or salt or co-crystal thereof, in combination with deep brain stimulation.

As used above, and throughout the description of the invention, the following terms, unless otherwise indicated, shall be understood to have the following meanings:

A “pharmaceutically acceptable carrier, diluent, or excipient” is a medium generally accepted in the art for the delivery of biologically active agents to mammals, e.g., humans.

A “dose” refers to a predetermined quantity or unit dose of mevidalen calculated to produce the desired therapeutic effect in a patient. As used herein “mg” refers to milligram. As used herein, dose ranges and doses provided of mevidalen represent the weight of the active pharmaceutical ingredient, 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone, regardless of the form in which it is provided, such as the free base, a cocrystalline form, or any other composition or form. Preferably unit doses are comprised of 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone and 4-hydroxybenzoic acid in cocrystalline form. The term “about” as used herein, means in reasonable vicinity of the stated numerical value, such as plus or minus 10% of the stated numerical value.

Methods of making and formulating mevidalen and/or 2-(2,6-dichlorophenyl)-1-((1S,3R)-5-(2-hydroxy-2-methylpropyl)-3-(hydroxymethyl)-1-methyl-3,4-dihydroisoquinolin-2(1H)-yl)ethan-1-one, are known in the art and recited for example in WO 2014/193781 and/or WO 2017/070068. Methods of preparing mevidalen and co-crystals thereof, and certain formulations and dosage forms thereof, are known to the skilled artisan, and are described in WO 2014193781 and/or WO 2017/070068. WO 2014/193781 discloses certain 3,4-dihydroisoquinolin-2(1H)-yl compounds as positive allosteric modulators (PAM) of the dopamine 1 receptor (D1), including 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone and a cocrystalline form comprising 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone and 4-hydroxybenzoic acid, and compositions thereof. WO 2017/070068 discloses crystalline 2-(2,6-dichlorophenyl)-1-[(1S,3R)-3-(hydroxymethyl)-5-(3-hydroxy-3-methylbutyl)-1-methyl-3,4-dihydroisoquinolin-2(1H)-yl]ethanone. Mevidalen is preferably formulated as pharmaceutical composition administered by any route which makes the compound bioavailable, including oral, intravenous, and transdermal routes. Most preferably, such pharmaceutical compositions are for oral administration. Mevidalen can be administered alone or in the form of a pharmaceutical composition with pharmaceutically acceptable carriers, diluents or excipients. Throughout the description, where compositions are described as having, including, or comprising specific components, it is contemplated that compositions also consist essentially of, or consist of, the recited components. Such pharmaceutical compositions and processes for making the same are known in the art (See, e.g., Remington: The Science and Practice of Pharmacy, L. V. Allen, Editor, 22nd Edition, Pharmaceutical Press, 2012). In a formulation mevidalen is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the formulations can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, gels, suppositories, sterile injectable solutions, and sterile packaged powders. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compounds of the invention can be formulated so as to provide quick, sustained or delayed release of the active ingredient after administration to the patient by employing procedures known in the art. One skilled in the art of preparing formulations can readily select the proper form and mode of administration depending upon the particular characteristics of the compound and/or form selected, the disorder or condition to be treated, the stage of the disorder or condition, and other relevant circumstances. The table below provides examples of selected unit dosage forms provided as tablets for oral administration according to the dosing regimens of the present invention. The skilled artisan can use these examples, along with readily known formulation methods, to provide additional formulations and/or unit dosage forms.

Unit Dose (mg mevidalen,
active ingredient)

| | 5 | 10 | 25 | 30 | 50 |
|---|---|---|---|---|---|
| Component | mg | mg | mg | mg | mg |
| Mevidalen - | 6.5 | 13.1 | 32.7 | 39.2 | 65.3 |
| Benzoic | | | | | |
| Acid Cocrystal | | | | | |
| (mg) | | | | | |
| Microcrystalline | 109.7 | 126.4 | 81.1 | 142.8 | 162.2 |
| Cellulose | | | | | |
| Croscarmellose | 6.3 | 7.5 | 6.3 | 10 | 12.5 |
| Sodium | | | | | |
| Sodium Stearyl | 2.5 | 3 | 2.5 | 4 | 5 |
| Fumarate | | | | | |
| Silixon Dioxide, | 0 | 0 | 2.5 | 4 | 5 |
| Core Tablet Weight | 125 | 150 | 125 | 200 | 250 |
| (mg): | | | | | |
| Coating: | 5 | 6 | 5 | 8 | 10 |

The unit doses of the present invention are formulated as pharmaceutical compositions administered by any route which makes the compound bioavailable, preferably such compositions are for oral administration. “Administration” or “administering”, as used herein, includes wherein the patient self-administers mevidalen, and/or wherein mevidalen is administered by another person, and/or wherein the patient is instructed and/or directed to consume mevidalen according to a particular regimen. Preferably mevidalen is administered in the morning. Preferably, mevidalen is taken daily. Preferably the indicated unit doses of mevidalen are taken daily, that is one time per day, as is indicated by the use of the term “per day”. As used herein, “daily administration” includes the administration of mevidalen as a specific treatment regimen intended to provide the beneficial effect from the long term and regular administration of mevidalen at the specified doses. In particular, “daily administration” includes administration every day consecutively for not less than twenty-one days in a row, or for as long as is needed to prevent the patients' signs and symptoms of a dopaminergic CNS disorder. If a patient misses an occasional day, then the patient may simply resume administration on the next day specified for administration, and such an instance would continue to represent “daily administration”. As used herein, “daily” means mevidalen is administered one time every 24-hour period, or one time every calendar day. As used herein, “daily” means mevidalen is administered on an ongoing consecutive basis, where administering includes as used herein includes both when the patient administers the doses, and/or wherein the patient is instructed to administer the doses as part of a treatment regimen. Where methods are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

Embodiments of the present disclosure include other dopamine D1 positive allosteric modulators, for instance as those described and/or exemplified in WO 2014193781 and/or WO 2017/070068, and WO 2019/204419, and WO 2016/055479, including any salts and/or co-crystals thereof. For instance, one such other dopamine D1 positive allosteric modulator is referred to as DPTQ, which is 2-(2,6-dichlorophenyl)-1-((1S,3R)-5-(2-hydroxy-2-methylpropyl)-3-(hydroxymethyl)-1-methyl-3,4-dihydroisoquinolin-2(1H)-yl)ethan-1-one, is shown below.

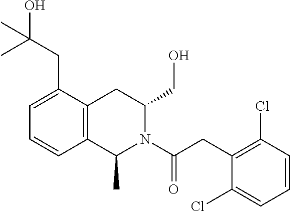 [SMILES: C[C@H]1c2cccc(CC(C)(C)O)c2C[C@H](CO)N1C(=O)Cc1c(Cl)cccc1Cl]

Embodiments of the present disclosure include other dopamine D1 positive allosteric modulators, for instance as those listed below, for instance a compound of the formula Ib:

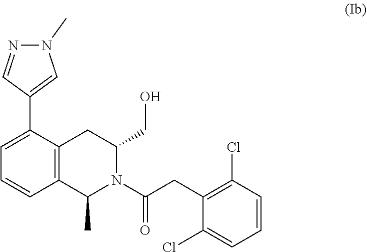 [SMILES: C[C@H]1c2cccc(-c3cnn(C)c3)c2C[C@H](CO)N1C(=O)Cc1c(Cl)cccc1Cl]

or a pharmaceutically acceptable salt or co-crystal thereof, which in the free base form can also be named as 2-(2,6-dichlorophenyl)-1-((1S,3R)-3-(hydroxymethyl)-1-methyl-5-(1-methyl-1H-pyrazol-4-yl)-3,4-dihydroisoquinolin-2(1H)-yl)ethan-1-one.

Embodiments of the present disclosure include other dopamine D1 positive allosteric modulators, for instance as those disclosed herein, or a pharmaceutically acceptable salt thereof or co-crystal thereof, and a pharmaceutically acceptable carrier, diluent or excipient.

Embodiments of the present disclosure include other dopamine D1 positive allosteric modulators, for instance as those listed below: The present invention provides a compound which is 2-(2,6-dichlorophenyl)-1-((1S,3R)-5-(1-ethyl-1H-pyrazol-4-yl)-3-(hydroxymethyl)-1-methyl-3,4-dihydroisoquinolin-2(1H)-yl)ethan-1-one, or a pharmaceutically acceptable salt thereof.

The present invention provides a compound which is 1-((1S,3R)-5-(1-cyclopropyl-1H-pyrazol-4-yl)-3-(hydroxymethyl)-1-methyl-3,4-dihydroisoquinolin-2(1H)-yl)-2-(2,6-dichlorophenyl)ethan-1-one, or a pharmaceutically acceptable salt thereof.

The present invention provides a compound which is 2-(2,6-dichlorophenyl)-1-((1S,3R)-3-(hydroxymethyl)-1-methyl-5-(1-(oxetan-3-yl)-1H-pyrazol-4-yl)-3,4-dihydroisoquinolin-2(1H)-yl)ethan-1-one, or a pharmaceutically acceptable salt thereof.

The present invention provides a compound which is 1-((1S,3R)-5-(1-(2-(11-oxidaneyl)ethyl)-1H-pyrazol-4-yl)-3-(hydroxymethyl)-1-methyl-3,4-dihydroisoquinolin-2(1H)-yl)-2-(2-chlorophenyl)ethan-1-one, or a pharmaceutically acceptable salt thereof.

The present invention provides a compound which is 2-(2-chloro-6-fluorophenyl)-1-((1S,3R)-3-(hydroxymethyl)-1-methyl-5-(1-(oxetan-3-yl)-1H-pyrazol-4-yl)-3,4-dihydroisoquinolin-2(1H)-yl)ethan-1-one, or a pharmaceutically acceptable salt thereof.

The present invention provides a compound which is 2-(2-chlorophenyl)-1-((1S,3R)-3-(hydroxymethyl)-1-methyl-5-(1-methyl-1H-pyrazol-4-yl)-3,4-dihydroisoquinolin-2(1H)-yl)ethan-1-one, or a pharmaceutically acceptable salt thereof.

The present invention provides a compound which is 2-(2,6-dichlorophenyl)-1-((1 S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2-chloro-6-fluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2,6-difluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2-chloro-5-fluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2-chloro-4-fluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2-fluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2,3-difluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

The present invention provides a compound which is 2-(2,5-difluorophenyl)-1-((1S,3R)-3-(hydroxymethyl)-1-methyl-5-(1H-pyrazol-4-yl)-3,4-dihydroisoquinolin-2(1H)-yl)ethan-1-one, or a pharmaceutically acceptable salt thereof.

Embodiments of the present disclosure includes dopamine D1 positive allosteric modulators above and/or salt or co-crystal thereof, and a pharmaceutically acceptable carrier, diluent or excipient.

As used herein, the term “patient” refers to a human, and patients to be treated by the present dosing regimens are early-stage Parkinson's patients, and preferably diagnosed but untreated Parkinson's patients, and as such share etiophathological aspects in that disturbances of dopamine signaling contribute to Parkinson's disease progression. Identification of patients with early-stage Parkinson's disease can be achieved by established methods known to the skilled artisan and described herein, for instance in Example 1.

In embodiments of the invention a patient is a human who has been diagnosed as having a medical risk for Parkinson's disease, prodromal Parkinson's disease, or early Parkinson's disease, in need of treatment with a D1 PAM, such as mevidalen, and using a dosing regimen described herein. In those instances where the disorders for which disease progression can be slowed, by the methods of the present disclosure are known by established and accepted classifications, such as prodromal Parkinson's disease, or newly diagnosed Parkinson's disease, or early-stage Parkinson's disease, their classifications can be found in various well-known medical texts. For example, at present, the 5th edition of the Diagnostic and Statistical Manual of Mental Disorders (DSM-5), provides a diagnostic tool for identifying many of the disorders described herein. Also, the International Classification of Diseases, Tenth Revision (lCD-10), provides classifications for many of the disorders described herein. The skilled artisan will recognize that there are alternative nomenclatures, nosologies, and classification systems for disorders described herein, including those as described in the DSM-5 and ICD-10, and that terminology and classification systems evolve with medical scientific progress. Parkinson's disease stages are well described and known to the skilled artisan, and the diagnostic methods described herein and as well as in the medical literature are used to identify patients in need of the present methods of treatment and prevention.

The terms “slowing the progression” as used herein refers to all processes wherein there may be a significant reduction, and/or more preferably freedom from significant signs and symptoms Parkinson's disease progression, as described herein, or known to the skilled artisan. The term “slowing the progression” of an existing disorder and/or symptoms thereof, does not necessarily indicate a total elimination of all symptoms.

The attending diagnostician, as one skilled in the art, can readily determine the dose chosen from the dosing regimens provided herein by observing results obtained from treatment. In determining a specific dose of Mevidalen from dosing regimens of the present invention, a number of factors are considered, including, but not limited to the weight, age, and general health of the patient; the degree of involvement or the severity of the disorder; the response of the individual patient; the use of other concomitant medication; and other relevant circumstances.

A dose regimen of the present invention may be used in combination with other drugs that are used in the treatment/prevention/suppression or amelioration of Parkinson's disease. Such other drug(s) may be administered, by a route and in an amount commonly used therefore, contemporaneously or sequentially with Mevidalen. For example, other active ingredients effective in the treatment of Parkinson's disease that may be combined with Mevidalen, include, but are not limited to dopamine agonists, dopamine precursors such as levodopa, COMT inhibitors, MAO-B inhibitors, and anti-cholinergics.

### BRIEF DESCRIPTION OF THE DRAWINGS

FIG. 1: Mevidalen treatment resulted in significant improvement in global functioning (ADCS-CGIC). In the PRESENCE study, participants with LBD, aged 40-85 years, and with a Hoehn and Yahr score of 0-4 and a Montreal Cognitive Assessment score of 10-23 were randomized 1:1:1:1 to mevidalen 10 mg, 30 mg, or 75 mg or placebo once-daily for 12 weeks (See Example 1). CGIC is a common method to evaluate clinical meaningfulness of interventions1. Mevidalen LS mean change from baseline to 12 weeks: −0.7 at 30 mg, −0.9 at 75 mg (p<0.001 for both). These results may reflect the multidimensional benefit of mevidalen.

### EXAMPLE 1: PHASE 2 CLINICAL STUDY FOR PARKINSON'S DISEASE DEMENTIA

### (Referred to as PRESENCE. NCT03305809)

The objective of this study was to assess the motor and non-motor effects of mevidalen in patients with Lewy Body Dementia (LBD) as measured by the MDS-UPDRS. Mevidalen is a positive allosteric modulator of D1 receptor (D1PAM). D1 receptor activation improves cognitive and motor function and enhances wakefulness in preclinical and clinical models. The Phase 2, 12-week PRESENCE study was designed to evaluate the symptomatic effect of mevidalen for treatment of cognition and other domains relevant to LBD including motor function, sleep, mood and apathy.

Participants with LBD, aged 40-85 years, and with a Hoehn and Yahr score of 0-4 and a Montreal Cognitive Assessment score of 10-23 were randomized 1:1:1:1 to mevidalen 10 mg, 30 mg, or 75 mg or placebo once-daily. The primary cognition outcome was CDR-CoA. Secondary outcomes included change from baseline to Week 12 in the MDS-UPDRS total score (sum of Parts I-III) and change in both the MDS-UPDRS Part II (motor experiences of daily living) and Part III (motor exam) scores. Analyses were also pre-specified for the Part I items of fatigue, daytime sleepiness, hallucinations, depressed mood and apathy and the Part IV items of motor fluctuations and dyskinesias.

Below is provided a protocol for study of mevidalen in PD with certain doses of the methods of treatment and dosing regimens described herein. The skilled artisan will be able to apply the teachings of this Example 1, and other disclosures provided herein, and conduct similar studies with additional doses and dosing regimens of the present invention.

Treatments for slowing the progression of Parkinson's disease, that are well tolerated, remains an unmet medical need. Mevidalen has for the first time as described herein been observed to improve signs and symptoms of Parkinson's disease in a relatively short period of chronic daily dosing (12 weeks). The PRESENCE study assessed three doses (10 mg, 30 mg, and/or 75 mg daily (or 50 mg based on interim analysis) (QD) oral dosing) of mevidalen vs. placebo over 12 weeks of treatment. The primary outcome was a measure of cognition and but also assessed additional endpoints described herein. PRESENCE was a randomized placebo-controlled trial in individuals with Parkinson's disease dementia to evaluate the safety and efficacy of (three doses of study drug) mevidalen in participants with mild-to-moderate Parkinson's disease dementia treated for 12 weeks.

Study HBEH included subjects who meet the revised MDS criteria for PD (Postuma et al. 2015) and mild-to-moderate dementia as defined by a decline in cognitive function, which in the opinion of the investigator has resulted in functional impairment and a MoCA score between 10 and 23 (Trzepacz et al. 2015). Per the revised MDS criteria, PDD can be diagnosed in the presence of dementia, regardless of the timing of dementia onset relative to PD diagnosis. Subjects diagnosed with dementia with Lewy bodies (DLBs) should be considered as also having PD if they meet the MDS PD criteria. Therefore, subjects may have dementia prior to, at the time of, or subsequent to the diagnosis of PD. Unlike registration trials of symptomatic therapies in PDD (Emre et al. 2004), the current study may include some subjects who would otherwise have met the traditional criteria (dementia prior to or within 1 year of motor onset) for DLB based on the timing of their dementia (Mckeith et al. 2005). This criterion was that the dementia occurs prior to or within 1 year of Parkinson's symptoms. The 1-year rule is arbitrary and based on the historical belief that PD was not associated with dementia; however, there is increasing controversy about the validity of this traditional approach to splitting the diagnoses (Berg et al. 2014). In support of the proposed approach, both disorders share a variety of clinical, genetic, and pathological features (Lippa et al. 2007; Postuma et al. 2009; Johansen et al. 2010). Both DLB and PDD are associated with similar impairments in cognition with predominant visuoperceptual abnormalities, improvement in memory with cues, and so on. Both are associated with prominent psychosis, neuroleptic sensitivity, and alterations in arousal. Prodromal features (e.g., rapid eye movement [REM] sleep behavior disorder, olfactory loss) are the same in both conditions. Non-motor symptoms with depression, anxiety, autonomic dysfunction and sleep disturbances occur with similar relative frequency in both. The same genetic mutations (alpha-synuclein duplications, glucocerebrosidase mutations) are associated with the development of either condition. Finally, they have a shared pathology with alpha-synuclein and Lewy body formation in the brain stem and cortex. Therefore, the Study HBEH meets current thinking about PDD and DLB that, apart from the timing of cognitive impairment, they are clinically and pathologically indistinguishable and would likely respond to similar therapeutic approaches (Aarsland et al. 2004; Ballard et al. 2006). Placebo is included as the control, in a blinded manner for investigator and site staff and subjects, to allow for an unbiased assessment of the safety data generated, which will allow for a more robust comparison between mevidalen and placebo data. Comparison of 3 dosage levels of mevidalen was chosen to evaluate dosage exposure response for safety and efficacy. Initial visits (Visit 3 to Visit 7) were selected to occur at a weekly interval to provide a detailed evaluation of the efficacy and safety of mevidalen during the initial treatment. A dosing duration of 12 weeks was selected, as it is estimated to be the minimum duration where a beneficial effect on cognition may be observed.

Primary objective was to test the hypothesis that mevidalen administered at 10 mg, 30 mg, and/or 75 mg daily (or 50 mg based on interim analysis) (QD) oral dosing for 12 weeks would result in a significant improvement in cognition in subjects with mild-to-moderate PDD compared with placebo. Primary endpoints are changes in the CoA composite score of the CDR-CCB from baseline to Week 12. Secondary objectives are decribed below.

| Secondary efficacy objectives | Secondary efficacy endpoints |
|---|---|
| To evaluate the global efficacy of | ADCS-CGIC score from baseline to Week |
| mevidalen | 12 |
| To evaluate the efficacy of mevidalen on | Change in the CDR-CCB PoA composite |
| cognitive outcomes | score from baseline to Week 12 |
| To evaluate the efficacy of mevidalen on | Change in the ADAS-Cog13 score from |
| cognitive outcomes | baseline to Week 12 |
| To evaluate the efficacy of mevidalen on | Change in the MoCA score from screening |
| cognitive outcomes | to Week 12 |
| To evaluate the efficacy of mevidalen on | Change in the NPI total and individual |
| neuropsychiatric symptoms | items scores from baseline to Week 12 |
| To evaluate the effect of mevidalen on | Change in the ESS score from baseline to |
| daytime sleepiness | Week 12 |
| To evaluate the effect of mevidalen on PD | Change in the MDS-UPDRS total score |
| severity | (sum of Parts I-III) from baseline to Week |
| | 12 |
| To evaluate the efficacy of mevidalen on | Change in the PDAQ-15 total score from |
| functional outcome | baseline to Week 12 |
| To evaluate the effect of mevidalen on | Change in D-KEFS Verbal Fluency test |
| verbal fluency | score from baseline to Week 12 |
| Safety objectives | Safety endpoints |
| To evaluate the effect of mevidalen on | Number of subjects who met the |
| acute changes of vital signs on the first day | potentially clinically significant vital signs |
| of dosing | criteria at 3 consecutive time points at Visit |
| | 3 (Day 1 stopping rules) |
| To evaluate the effect of mevidalen on SBP | Change in in-clinic SBP from 0 up to 8 |
| on the first day of dosing | hours post dose on the first day of study |
| | drug dosing |
| To evaluate the effect of mevidalen on | Change in in-clinic pulse rate from 0 up to |
| pulse rate on the first day of dosing | 8 hours post dose on the first day of study |
| | drug dosing |
| To evaluate the effect of mevidalen on SBP | Change in in-clinic mean SBP at baseline |
| from baseline to Week 12 | to mean SBP at Week 12 |
| To evaluate the effect of mevidalen on | Change in in-clinic mean pulse rate at |
| pulse rate from baseline to Week 12 | baseline to mean pulse rate at Week 12 |

| Pharmacokinetics objectives: | Pharmacokinetics objectives: |
|---|---|
| To assess the PK of mevidalen in a | Steady-state trough plasma concentrations |
| population of subjects with mild-to- | of mevidalen at Week 12 |
| moderate dementia due to PD | |

Abbreviations: ADAS-Cog13=13—item Alzheimer's Disease Assessment Scale—Cognitive subscale; ADCS-CGTC=Alzheimer's Disease Cooperative Study—Clinician Global Impression of Change; CDR-CCB=Cognitive Drug Research—Computerized Cognition Battery; CoA=Continuity of Attention; D-KEFS=Delis-Kaplan Executive Function System; ESS=Epworth Sleepiness Scale; MDS-UPDRS=Movement Disorder Society's Unified Parkinson's Disease Rating Scale; MoCA=Montreal Cognitive Assessment; NPI=Neuropsychiatric Inventory; PD=Parkinson's disease; PDD=Parkinson's disease dementia; PDAQ-15=Penn Parkinson's Daily Activities Questionnaire-15; PK=pharmacokinetics; PoA=Power of Attention; QD=once a day; SBP=systolic blood pressure.

### REFERENCES

Postuma R B, Berg D, Stern M, Poewe W, Olanow C W, Oertel W, Obeso J, Marek K, Litvan I, Lang A E, Halliday G, Goetz C G, Gasser T, Dubois B, Chan P, Bloem B R, Adler C H, Deuschl G. MDS clinical diagnostic criteria for Parkinson's disease. Mov Disord. 2015; 30(12):1591-1601.
Trzepacz P T, Hochstetler H, Wang S, Walker B, Saykin A J; Alzheimer's Disease Neuroimaging Initiative. Relationship between the Montreal Cognitive Assessment and Mini-mental State Examination for assessment of mild cognitive impairment in older adults. BMC Geriatr. 2015; 15:107.
Emre M, Aarsland D, Albanese A, Byrne E J, Deuschl G, De Deyn P P, Durif F, Kulisevsky J, van Laar T, Lees A, Poewe W, Robillard A, Rosa M M, Wolters E, Quarg P, Tekin S, Lane R. Rivastigmine for dementia associated with Parkinson's disease. N Engl J Med. 2004; 351(24):2509-2518.
McKeith I G, Dickson D W, Lowe J, Emre M, O'Brien J T, Feldman H, Cummings J, Duda J E, Lippa C, Perry E K, Aarsland D, Arai H, Ballard C G, Boeve B, Burn D J, Costa D, Del Ser T, Dubois B, Galasko D, Gauthier S, Goetz C G, Gomez-Tortosa E, Halliday G, Hansen L A, Hardy J, Iwatsubo T, Kalaria R N, Kaufer D, Kenny R A, Korczyn A, Kosaka K, Lee V M, Lees A, Litvan I, Londos E, Lopez O L, Minoshima S, Mizuno Y, Molina J A, Mukaetova-Ladinska E B, Pasquier F, Perry R H, Schulz J B, Trojanowski J Q, Yamada M; Consortium on DLB. Diagnosis and management of dementia with Lewy bodies: third report of the DLB Consortium. Neurology. 2005; 65(12):1863-1872.
Berg D, Postuma R B, Bloem B, Chan P, Dubois B, Gasser T, Goetz C G, Halliday G M, Hardy J, Lang A E, Litvan I, Marek K, Obeso J, Oertel W, Olanow C W, Poewe W, Stern M, Deuschl G. Time to redefine PD? Introductory statement of the MDS Task Force on the definition of Parkinson's disease. Mov Disord. 2014; 29(4):454-462.
Lippa C F, Duda J E, Grossman M, Hurtig H I, Aarsland D, Boeve B F, Brooks D J, Dickson D W, Dubois B, Emre M, Fahn S, Farmer J M, Galasko D, Galvin J E, Goetz C G, Growdon J H, Gwinn-Hardy K A, Hardy J, Heutink P, Iwatsubo T, Kosaka K, Lee V M, Leverenz J B, Masliah E, McKeith I G, Nussbaum R L, Olanow C W, Ravina B M, Singleton A B, Tanner C M, Trojanowski J Q, Wszolek Z K; DLB/PDD Working Group. DLB and PDD boundary issues: diagnosis, treatment, molecular pathology, and biomarkers. Neurology. 2007; 68(11):812-819.
Postuma R B, Gagnon J F, Vendette M, Montplaisir J Y. Idiopathic REM sleep behavior disorder in the transition to degenerative disease. Mov Disord. 2009; 24(15):2225-2232.
Johansen K K, White L R, Sando S B, Aasly J O. Biomarkers: Parkinson disease with dementia and dementia with Lewy bodies. Parkinsonism Relat Disord. 2010; 16(5):307-315.
Aarsland D, Ballard C G, Halliday G. Are Parkinson's disease with dementia and dementia with Lewy bodies the same entity? J Geriatr Psychiatry Neurol. 2004; 17(3):137-145. American Psychiatric Association. Diagnostic and statistical manual of mental disorders. 5th ed. Washington, DC; 2013.
Ballard C, Ziabreva I, Perry R, Larsen J P, O'Brien J, McKeith I, Perry E, Aarsland D. Differences in neuropathologic characteristics across the Lewy body dementia spectrum. Neurology. 2006; 67(11):1931-1934.
Yesavage J A, Brink T L, Rose T L, Lum O, Huang V, Adey M., Leirer V O. Development and validation of a geriatric depression screening scale: A preliminary report. J Psychiatr Res. 1983; 17:37-49.
Sheikh J I, Yesavage J A. Geriatric Depression Scale (GDS): Recent evidence and development of a shorter version. Clin Gerontologist. 1986; 5:165-173.

Summary of Study Design: Study 17S-MC-HBEH (HBEH) is a multicenter, randomized, double-blind, parallel-group, placebo-controlled, fixed-dosage, Phase 2a study comparing 3 dosages of mevidalen (10, or 30, or 75 mg administered orally [or 50 mg based on interim analysis] once a day [QD]) with placebo over 12 weeks in subjects with mild-to-moderate PDD. The study includes a Screening Period (Visits 1 to 2) of a minimum of 7 days and up to 14 days, a Pretreatment Period of a minimum of 11 days and up to 17 days (Visits 2 to 3), a 12-week Treatment Period (Visits 3 to 11), and a 14-day Safety Follow-Up Period (Visits 11 to 801 or early termination [ET]/discontinuation [DC] visit to Visit 801). Subjects who meet entry criteria will be randomized in a 1:1:1:1 ratio to mevidalen (10 or 30 or 75 mg QD) or placebo. The primary objective of the study is to test the hypothesis that mevidalen administration for 12 weeks will result in a significant improvement in cognition as measured by the change from baseline to Week 12 in the Continuity of Attention (CoA) composite score of the Cognitive Drug Research Computerized Cognition Battery (CDR-CCB), in subjects with mild-to-moderate PDD, compared to placebo. The CoA has demonstrated a significant treatment effect in previous trials in subjects with PDD (Wesnes et al. 2005; Rowan et al. 2007). Treatment Arms and Duration: Study HBEH involves a comparison of mevidalen 10 mg, 30 mg, and 75 mg (or 50 mg based on interim analysis) administered orally QD with placebo over 12 weeks. Number of subjects: Approximately 400 subjects will be screened to achieve 340 randomized and an estimated total of 85 evaluable subjects per treatment group.

### Statistical Analysis:

Efficacy Analysis: All subjects in the evaluable patient population (EPP) will be considered for the efficacy analysis. The primary analysis on CoA will occur when all subjects complete 12 weeks of treatment. The analysis of CoA will utilize a Bayesian MMRM model. The Bayesian analysis may use uninformative priors for all terms in the model. These will be diffuse Normal distributions centered on zero. Priors for variance will follow an inverse gamma distribution. Further details of the Bayesian analysis will be provided in the SAP. The MMRM model will account for longitudinal data assessed throughout the study, after 1, 2, 4, 6, 8, 10, and 12 weeks of dosing. The change of CoA from the baseline to Week 12 will be the dependent variable. The model will comprise fixed (baseline value, treatment, visit) and random effects (subject) and the interaction terms (treatment by visit, baseline value by visit). Unstructured variance structure will be applied in the model, but if it fails to converge, other suitable structures will be investigated. The primary comparison will be the contrast (difference in least squares mean) between treatments and placebo for the Week 12 change from baseline. The secondary efficacy outcomes: the change from baseline at 12-week time point of total scores (or composite values) of Alzheimer's Disease Cooperative Study—Clinician Global Impression of Change (ADCS-CGIC), CDR-CCB Power of Attention (PoA), 13-item Alzheimer's Disease Assessment Scale—Cognitive subscale (ADAS-Cog13),

Montreal Cognitive Assessment (MoCA), Neuropsychiatric Inventory (NPI), Epworth Sleepiness Scale (ESS), Movement Disorder Society's Unified Parkinson's Disease Rating Scale (MDS-UPDRS), Penn Parkinson's Daily Activities Questionnaire 15 (PDAQ-15), and Delis-Kaplan Executive Function System (D-KEFS) will follow the same analysis method as above. Missing records in some scales (e.g., ADAS-Cog) will be imputed as detailed in the statistical analysis plan. For the scales where the imputation is not done, if any item is missing, any total or sum involving that item will be considered missing. No adjustment for multiple comparisons will be made.

Safety Analysis: Safety analyses are based on the safety population and analysis will include listings and/or summaries of the following: adverse events (AEs), serious adverse events (SAEs), treatment-emergent adverse events (TEAEs), laboratory measures, vital signs, electrocardiogram readings and number of subjects who met the potentially clinically significant vital signs criteria at 3 consecutive time points at Visit 3 (Day 1 stopping rules). Mixed-model repeated measures analysis will be used to compare the change in in-clinic blood pressure (BP) and pulse rate from pretreatment up to 8 hours post dose measured on the first day of study drug dosing (V3). Two baselines will be considered in the change from baseline analyses: the V3 pretreatment value and the time-matched baselines from Visit 2 (hourly value 0 to 6 hours). For the second baseline, the V3, 7- and 8-hour time points will use the V2 6-hour time point as their baseline value. A separate change from baseline analysis will be completed for each baseline approach. Mixed-model repeated measures analyses will also be used to compare change in in-clinic BP and pulse rate from V2 (daily average 0 to 6 hours) to Week 6/Visit 8 and Week 12/Visit 11 (daily average 0 to 6 hours), to evaluate the change in BP and pulse rate over 12 weeks of dosing.

Pharmacokinetics (PK): Pharmacokinetic analyses will be conducted on subjects who receive at least 1 dose of the study drug and have 1 measurable concentration. A model-based approach may be implemented using nonlinear mixed effects modeling (NONMEM) or other appropriate software to estimate PK parameters.

Additional endpoints and biomarker data collected during the study may be evaluated in an exploratory manner.

Interim Analysis: Safety interim analyses will be conducted on the number of subjects on each treatment who met the potentially clinically significant vital signs criteria at 3 consecutive time points at Visit 3 (Day 1 stopping rules). This will be done after 50, 100, and 150 subjects have completed Visit 3. If there is >60% probability that the difference in rate of subjects meeting Day 1 stopping rules for 75 mg mevidalen compared to placebo is >0.3, the 75-mg dose level will be replaced with 50 mg for the subsequently enrolled subjects. Those already on 75-mg dose and passed the Day 1 stopping rules will remain on 75 mg. In the event of an unacceptable rate of subjects meeting Day 1 stopping rules at other doses, adjustments to doses may be made for subsequently randomized subjects at the discretion of the Internal Assessment Committee (IAC). Additional efficacy analyses may be conducted at the time of these safety interim analyses. A safety and efficacy interim analysis will be conducted when 170 randomized subjects have completed Visit 11 (Week 12) assessments. All potential efficacy analyses may be used for internal decision making, but are not planned to stop the study. Study HBEH will include men and women aged 40 to 85 years with mild-to-moderate PDD. Subjects are eligible to be included in the study only if they meet all the following criteria at enrollment (Visit 1) (note that inclusion criteria [6] to [10] must be met or at an additional visit[s]):

Type of Subject and Disease Characteristics: [1] Male and female subjects aged 40 to 85 years (inclusive). [2] Have idiopathic PD per MDS criteria (Postuma et al. 2015) with at least 2 years of PD symptoms. [3] Have dementia as defined by a decline in cognitive function, which in the opinion of the investigator has resulted in functional impairment. [4] Have a MoCA score of 10 to 23 at the time of screening. [5] Are Modified Hoehn and Yahr Stages 1 to 4. [6] Have a BP or pulse rate at Visit 1 and Visit 3, as determined by 3 sequential BP/pulse rate measurements in the seated position:

For Subjects <60 years old: a mean systolic blood pressure (SBP) less than or equal to 140 mmHg, a mean diastolic BP less than or equal to 90 mmHg, and a mean pulse rate less than or equal to 90 beats/min in the seated position, and each of the 3 SBP measurement must be less than 180 mmHg.

For Subjects >60 years old: a mean SBP less than or equal to 150 mmHg, a mean diastolic BP less than or equal to 90 mmHg, and a mean pulse rate less than or equal to 90 beats/min in the seated position, and each of the 3 SBP measurement must be less than 180 mmHg.

The following PD severity and cognitive assessments, as well as the Columbia-Suicide Severity Rating Scale (C-SSRS), will be done at Visit 1 as part of the subject eligibility evaluation: Movement Disorder Society (MDS) Clinical Diagnostic Criteria for Parkinson's disease Enrolled individuals will meet MDS criteria for clinically probable PD (Postuma et al. 2015). Subjects must have bradykinesia with either rest tremor and/or rigidity. Subjects must not have any absolute exclusion criteria described in Appendix 5. Subjects must not have the presence of greater than 2 red flags; if 1 red flag is present then it must be offset by 1 supportive criterion and if 2 red flags are present it must be offset by 2 supportive criteria.

In addition to meeting criteria for PD, subjects must meet criteria for dementia as described below (Montreal Cognitive Assessment [MoCA] Scale). The MDS criteria do not consider dementia as an exclusion criterion for PD and therefore there will be no restriction on the timing of dementia relative to the development of the motor features of PD.

Modified Hoehn and Yahr Scale: Enrolled individuals must be Hoehn and Yahr Stage 1 to Stage 4 at screening. The Hoehn and Yahr Scale (Hoehn and Yahr 1967) is used to describe the symptom progression of PD. The scale was originally described in 1967 and included Stages 1 through 5. It has since been modified with the addition of Stages 1.5 and 2.5 to account for the intermediate course of PD. The modified Hoehn and Yahr scale is as follows: Stage 0: No signs of disease, Stage 1: Unilateral disease, Stage 1.5: Unilateral plus axial involvement, Stage 2: Bilateral disease, without impairment of balance, Stage 2.5: Mild bilateral disease, with recovery on pull test, Stage 3: Mild-to-moderate bilateral disease; some postural instability; physically, independent, Stage 4: Severe disability; still able to walk or stand unassisted, Stage 5: Wheelchair bound or bedridden unless aided.

Montreal Cognitive Assessment Scale: Enrolled individuals must have a MoCA score of 10 to 23 at screening.

Geriatric Depression Scale: Enrolled individuals must have a Geriatric Depression Scale—Short Form (GDS-S) score of ≤6 at screening. The GDS is a site-administered questionnaire of depression in older adults (Yesavage et al. 1983). Users respond in a “Yes/No” format. Originally developed as a 30-item scale (Long Form), it has since been shortened to a 15-item scale (Short Form), which can be completed in approximately 5 to 7 minutes (Sheikh and Yesavage 1986). Of the 15 items, 10 are indicative of depression when answered “Yes” and 5 are indicative of depression when answered “No.”

Columbia-Suicide Severity Rating Scale—Children's Version: The C-SSRS is a scale that captures the occurrence, severity, and frequency of suicide-related thoughts and behaviors during the corresponding assessment period. The C-SSRS, included here as a screening assessment, is described in detail in Section 9.4.4. The C-SSRS “Baseline” version will be used at screening, and the findings will constitute the baseline assessment. The C-SSRS will be administered to the subject after the cognitive and functional assessments. Responses from subject will be considered when administering the scale. If it is determined that the subject has suicidal ideation or behavior at this baseline assessment, then the subject will not be randomized and will be discontinued from the study.

### Dose Dependent Effect of Mevidalen on Motor and Non-Motor Endpoints Observed in PRRESENCE.

The Phase 2, 12-week PRESENCE study was designed to evaluate the symptomatic effect of mevidalen for treatment of cognition and other domains relevant to LBD including motor function, sleep, mood and apathy. Participants with LBD aged 40-85 years, and with a Hoehn and Yahr score of 0-4 and a Montreal Cognitive Assessment score of 10-23 were randomized 1:1:1:1 to mevidalen 10 mg, 30 mg, or 75 mg or placebo once-daily. The primary cognition outcome was CDR-CoA. Secondary outcomes included change from baseline to Week 12 in the MDS-UPDRS total score (sum of Parts I-III) and change in both the MDS-UPDRS Part II (motor experiences of daily living) and Part III (motor exam) scores. Analyses were also pre-specified for the Part I items of fatigue, daytime sleepiness, hallucinations, depressed mood and apathy and the Part IV items of motor fluctuations and dyskinesias.

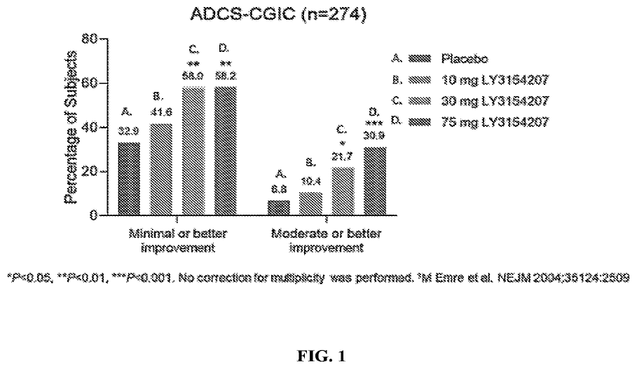

While mevidalen did not improve the primary cognitive outcome, there was a significant, dose-dependent improvement in the MDS-UPDRS total score. Compared to placebo, for 10, 30 and 75 mg dose the LS mean change in mevidalen was −6.58 (p=0.026), −7.56 (p=0.014) and −10.77 (p<0.001), respectively. There was a dose-dependent improvement in MDS-UPDRS Part II, significant for the 30 and 75 mg dose (p=0.014 and p<0.001, respectively) and Part 111, significant for the 75 mg dose (p=0.032). Individual item analysis revealed significant improvements in the Part I items of fatigue and daytime sleepiness at all doses, significant improvement in multiple items on Part II, significant improvement in the Part III bradykinesia items (all doses) and rigidity items (significant for mevidalen 30 mg); and a dose-dependent worsening of dyskinesias items in Part IV, significant for mevidalen 75 mg. Notably, mevidalen treatment resulted in significant improvement in global functioning (ADCS-CGIC) (See FIG. 1).

Mevidalen, which harnesses a novel mechanism of action (D1 PAM), resulted in symptomatic improvement of motor and select non-motor symptoms of parkinsonism associated with LBD, and avoiding worsening of non-motor symptoms associated with traditional dopaminergic therapy. Mevidalen harnesses a novel mechanism of action (D1 PAM) that may have the potential, preferably when used in prodromal or early-stage patients in particular, to slow or delay disease progression.

The PRESENCE trial also provided evidence that a cardiovascular effect is observed reflecting an acute increase in blood pressure and heart rate, and accommodation of these effects at lower doses of 10 and 30 mg. However, the adverse event profile and cardiovascular effects at the 75 mg dose may limit the clinical utility of higher doses. There, a preferred dosing regimen of the present invention is 10 mg to 50 mg daily for slowing or delaying the progression of Parkinson's disease.

Deep brain stimulation (DBS) implanted in early-stage Parkinson disease (PD) was found to decrease the risk of disease progression and lessen the need for multiple, simultaneous prescription drugs, according to study findings published in Neurology (Hacker M L, Turchan M, Heusinkveld L E, et al. Deep brain stimulation in early-stage Parkinson disease: five year outcomes. Neurol. Jul. 28, 2020; 95 (4)). The authors conclude the results suggest that early DBS reduces the need for and complexity of PD medications while providing long-term motor benefit over standard medical therapy. The Food and Drug Administration approved the conduct of a prospective, multicenter, pivotal clinical trial of DBS in early-stage PD (IDEG050016). Deep brain stimulation (DBS) is a well-established treatment in PD. Clinical trials have shown DBS improves motor symptoms, fluctuations, and quality of life, compared with medical therapy alone. The target is usually in the subthalamic nucleus (STN-DBS) or in the globus pallidus internus (STN-GPi) with similar motor benefits, whereas thalamic DBS is also an option to treat tremors. Surgical treatment tends to be considered when motor fluctuations and dyskinesias become disabling, despite motor features continuing to respond to 1-dopa. Surgical strategies used to be evaluated after 10-13 years of PD diagnosis. A multicenter randomized trial has showed quality of life might be improved after STN-DBS in comparison with optimal medical therapy, if DBS was performed in an early stage of disease. Despite the effectiveness of DBS, dopaminergic-resistant symptoms (i.e., axial symptoms), which poorly respond to this technique, can be present. Therefore, new targets, such as pedunculopontine nucleus, substantia nigra and thalamus, have been emerging as treatment of motor features.

The present disclosure provides the concept that treatment with mevidalen, and/or D1 PAM agents as described herein, preferably in the early stages of PD, can provide a means to pharmacologically enhance dopaminergic signally, and similar to DBS, can provide a means to achieve at least some of the benefits of DBS, without surgical interventions, and when applied in earlier stages may slow or inhibit or mitigate at least some facets of Parkinson's disease progression in some patients. Mevidalen and D1 PAM's, according to the methods and embodiments as described herein, can also be used in combination with DBS to slow the progression of Parkinson's disease.

Administration of mevidalen as described herein provides a novel approach to slow or prevent the progression of Parkinson's disease in one or more aspect of disease progression. The present methods of treatment using mevidalen are conceived to slow or prevent the onset and/or progression of Parkinson's disease in one or more aspect of disease progression preferably in prodromal stage patients as described herein and known to the skilled artisan, slowing the progression to Early-stage symptoms. The present methods of treatment using mevidalen are conceived to slow or prevent the progression of Parkinson's disease in one or more aspect of disease progression preferably in Early-stage patients as described herein, slowing the progression to Advanced stage symptoms. The present methods of treatment using mevidalen are conceived to slow or prevent the progression of Parkinson's disease in one or more aspect of disease progression preferably, in Advanced stage patients as described herein, slowing the progression to Complicated stage symptoms. The methods described herein are particularly useful for patients having been identified and/or diagnosed as being at risk of, and/or having the aforementioned stages of, Parkinson's disease, wherein the patient has further been diagnosed as experiencing one or more signs and symptoms of Parkinson's disease risk, or Parkinson's disease symptoms. Means to slow Parkinson's disease progression, using the mevidalen and D1 PAM agents disclosed herein, are expected to provide a variety of possible real world clinical benefits for care of the affected patients, including decreased caregiver burden, increased quality of life, and potentially delayed progression to nursing home care or severe dementia.

1-19. (canceled)

20. A method of slowing progression of Parkinson's Disease in a patient in need thereof, comprising administering to said patient a dopamine D1 positive allosteric modulator, or salt or co-crystal thereof.

21. The method of claim 20, wherein the patient has prodromal Parkinson's disease.

22. The method of claim 20, wherein the patient has Early-stage Parkinson's disease.

23. The method of claim 20, wherein the patient has Advanced stage Parkinson's disease.

24. The method according to claim 20, wherein patient is previously untreated for Parkinson's disease.

25. A method according to claim 20, wherein the D1 positive allosteric modulator is mevidalen, or a pharmaceutically acceptable co-crystal thereof.

26. A method according to claim 25, wherein the mevidalen, or a pharmaceutically acceptable co-crystal thereof, is orally administered daily in a dose of 5 to 60 mg per dose.

27. A method according to claim 25, wherein the mevidalen, or a pharmaceutically acceptable co-crystal thereof, is orally administered daily in a dose of 10 to 50 mg per dose.

28. A method according to claim 25, wherein the mevidalen, or a pharmaceutically acceptable co-crystal thereof, is orally administered daily in a dose selected from the group consisting of 10 mg, 15 mg, 20 mg, 25 mg, 30 mg, 35 mg, 40 mg, 45 mg, and 50 mg, per dose.

29. A method according to claim 28, wherein the mevidalen, or a pharmaceutically acceptable co-crystal thereof, is orally administered daily in a dose of 50 mg per dose.

30. A method according to claim 28, wherein the mevidalen, or a pharmaceutically acceptable co-crystal thereof, is orally administered daily in a dose of 45 mg per dose.

31. A method according to claim 28, wherein the mevidalen, or a pharmaceutically acceptable co-crystal thereof, is orally administered daily in a dose of 40 mg per dose.

32. A method according to claim 28, wherein the mevidalen, or a pharmaceutically acceptable co-crystal thereof, is orally administered daily in a dose of 35 mg per dose.

33. A method according to claim 28, wherein the mevidalen, or a pharmaceutically acceptable co-crystal thereof, is orally administered daily in a dose of 30 mg per dose.

34. A method according to claim 28, wherein the mevidalen, or a pharmaceutically acceptable co-crystal thereof, is orally administered daily in a dose of 25 mg per dose.

35. A method according to claim 28, wherein the mevidalen, or a pharmaceutically acceptable co-crystal thereof, is orally administered daily in a dose of 20 mg per dose.

36. A method according to claim 28, wherein the mevidalen, or a pharmaceutically acceptable co-crystal thereof, is orally administered daily in a dose of 15 mg per dose.

37. A method according to claim 28, wherein the mevidalen, or a pharmaceutically acceptable co-crystal thereof, is orally administered daily in a dose of 10 mg per dose.
